SEQUENCE LISTING (28 sequences)

SEQ ID NO: 1 - RNA, 52 nt, synthetic construct
  source 1..52: mol_type = other RNA
  misc_feature 1: note = Contains a triphosphate bridge
ggggccattattcaacgggaaacgtacgcacgaggccgcgattaaattccaa

SEQ ID NO: 2 - RNA, 24 nt, synthetic construct
  source 1..24: mol_type = other RNA
cggcctcgtgcgtgacgtttcccg

SEQ ID NO: 3 - RNA, 29 nt, synthetic construct
  source 1..29: mol_type = other RNA
  misc_feature 1: note = Contains a triphosphate bridge
ggggccattattcaacgggaaacgtcacg

SEQ ID NO: 4 - RNA, 28 nt, synthetic construct
  source 1..28: mol_type = other RNA
gggccattattcaacgggaaacgtcacg

SEQ ID NO: 5 - RNA, 39 nt, synthetic construct
  source 1..39: mol_type = other RNA
gcttgttctttttgcacaagctcagaataaacgcnnnnn

SEQ ID NO: 6 - RNA, 19 nt, synthetic construct
  source 1..19: mol_type = other RNA
ttctgcaaaaagaacaagc

SEQ ID NO: 7 - RNA, 30 nt, synthetic construct
  source 1..30: mol_type = other RNA
nnttgcagaagctcagaataaacgcnnnnn

SEQ ID NO: 8 - RNA, 19 nt, synthetic construct
  source 1..19: mol_type = other RNA
gcgtttattctgagcttct

SEQ ID NO: 9 - RNA, 20 nt, synthetic construct
  source 1..20: mol_type = other RNA
gcgtttattctgagcttctg

SEQ ID NO: 10 - RNA, 41 nt, Homo sapiens
  source 1..41: mol_type = other RNA
gactcttctggtccccacagactcagagagaacccaccatg

SEQ ID NO: 11 - RNA, 24 nt, synthetic construct
  source 1..24: mol_type = other RNA
catggtgggttctctctgagtctg

SEQ ID NO: 12 - RNA, 23 nt, synthetic construct
  source 1..23: mol_type = other RNA
atggatgggttctctctgagtct

SEQ ID NO: 13 - RNA, 23 nt, synthetic construct
  source 1..23: mol_type = other RNA
catggtgggttctctctgagtct

SEQ ID NO: 14 - RNA, 23 nt, synthetic construct
  source 1..23: mol_type = other RNA
atggtgggttctctctgagtctg

SEQ ID NO: 15 - RNA, 24 nt, synthetic construct
  source 1..24: mol_type = other RNA
catggtgggttctctctgagtctg

SEQ ID NO: 16 - RNA, 23 nt, synthetic construct
  source 1..23: mol_type = other RNA
catggtgggttctctctgagtct

SEQ ID NO: 17 - RNA, 22 nt, synthetic construct
  source 1..22: mol_type = other RNA
catggtgggttctctctgagtc

SEQ ID NO: 18 - RNA, 21 nt, synthetic construct
  source 1..21: mol_type = other RNA
catggtgggttctctctgagt

SEQ ID NO: 19 - RNA, 54 nt, Homo sapiens
  source 1..54: mol_type = other RNA
gacatttgcttctgacacaactgtgttcactagcaacctcaaacagacaccatg

SEQ ID NO: 20 - RNA, 24 nt, synthetic construct
  source 1..24: mol_type = other RNA
tgtttgaggttgctagtgaacaca

SEQ ID NO: 21 - RNA, 24 nt, synthetic construct
  source 1..24: mol_type = other RNA
ctgtttgaggttgctagtgaacac

SEQ ID NO: 22 - RNA, 24 nt, synthetic construct
  source 1..24: mol_type = other RNA
tctgtttgaggttgctagtgaaca

SEQ ID NO: 23 - RNA, 24 nt, synthetic construct
  source 1..24: mol_type = other RNA
gtctgtttgaggttgctagtgaac

SEQ ID NO: 24 - RNA, 18 nt, synthetic construct
  source 1..18: mol_type = other RNA
ctgagcttctgcaaaaag

SEQ ID NO: 25 - RNA, 18 nt, synthetic construct
  source 1..18: mol_type = other RNA
gcgtttattctgagttct

SEQ ID NO: 26 - RNA, 20 nt, synthetic construct
  source 1..20: mol_type = other RNA
cgtttattctgagcttctgc

SEQ ID NO: 27 - RNA, 20 nt, synthetic construct
  source 1..20: mol_type = other RNA
gtttattctgagcttctgca

SEQ ID NO: 28 - RNA, 21 nt, synthetic construct
  source 1..21: mol_type = other RNA
gtttattctgagcttctgcaa

(19) United States
(12) Patent Application Publication
(10) Pub. No.: US 2026/0250747 A1
(43) Pub. Date: Aug. 27, 2026

(54) ANALYSIS OF MRNA 5' CAPPING EFFICIENCY

(71) Applicant: CellScript, LLC, Madison, WI (US)

(72) Inventors: Jerome J. Jendrisak, Middleton, WI (US); Ronald J. Meis, Brooklyn, WI (US); Daniel Gerhardt, Madison, WI (US); Kari Weber, Middleton, WI (US); Gary A. Dahl, Madison, WI (US)

(21) Appl. No.: 19/551,252

(22) Filed: Feb. 26, 2026

Related U.S. Application Data
(60) Provisional application No. 63/763,639, filed on Feb. 26, 2025.

(51) Int. Cl. C12Q 1/683; C12Q 1/6806

(52) U.S. Cl. CPC C12Q 1/683; C12Q 1/6806

(57) ABSTRACT

The invention relates to methods of quantitating the relative amounts or percentages of RNA molecules that are 5′-capped and that are uncapped in an RNA sample or for quantifying the efficiency of capping of an in vitro-synthesized mRNA of interest that has been subjected to a capping enzyme system. The invention further relates to kits and kit-of-parts for use in said methods and the use of a nuclease that selectively cleaves RNA in a DNA/RNA hybrid in said methods.

### SEQUENCE LISTING

The text of the computer readable sequence listing filed herewith, titled “44810-202_SEQUENCE_LISTING”, created Feb. 26, 2026, having a file size of 26,565 bytes, is hereby incorporated by reference in its entirety.

### FIELD OF THE INVENTION

The invention relates to methods of quantitating the relative amounts or percentages of RNA molecules that are 5′-capped and that are uncapped in an RNA sample or for quantifying the efficiency of capping of an in vitro-synthesized mRNA of interest that has been produced in by in vitro transcription (IVT) with an RNA polymerase in a reaction mixture comprising a cap analog and NTPs by post-transcriptional capping of IVT-produced RNA using a capping enzyme system. The invention further relates to kits and kit-of-parts for use in said methods and the use of a nuclease that selectively cleaves RNA in a DNA/RNA hybrid in said methods.

### BACKGROUND OF THE INVENTION

Messenger RNA (mRNA) is a crucial molecule that transmits the genetic information stored in DNA to ribosomes, the protein-making machinery of cells. 5′-capping ensures that the mRNA is efficiently read during protein translation and reduces the induction of an innate immune response.

In eukaryotes, naturally occurring caps are composed of 7-methylguanosine linked via a 5′-5′ triphosphate bridge (m7GTP) to mRNAs. These 5′ caps are recognized in cells by the eukaryotic translation initiation factor 4E (eIF4e) and are crucial for the function of mRNAs in cells as uncapped mRNAs will not be efficiently translated. A variety of cap analogs have been developed in efforts to allow for better capping efficiency, enhanced translation, reduced intracellular de-capping and/or reduced immunogenicity. These include by are not limited to anti-Reverse Cap Analog (ARCA), unlocked nucleic acid (UNA) caps, Photoactivatable cap analogs, 3′Acm caps and dinucleotide/trinucleotide caps, such as described by Masahide Ishikawa, et al, in: “Preparation of eukaryotic mRNA having differently methylated adenosine at the 5′-terminus and the effect of the methyl group in translation”, Nucleic Acids Symposium Series, Vol 53, Issue 1, September-October 2009, pp 129-130, doi.org/10.1093/nass/nrp065 (and in the references therein). No cap analog results in mRNA production with 100% capping efficiency, so the evaluation of mRNA capping efficiency remains important prior to evaluation of mRNAs experimentally.

Current pharmaceutical industry recommendations for evaluation of mRNA 5′ capping efficiency include reverse-phase liquid chromatography mass spectrometry (RP-LC-MS/MS), ion pair reversed-phase high-performance liquid chromatography (IP-RP-HPLC), or liquid chromatography mass spectrometry (LC-MS/MS). In this regard, see “Analytical Procedures for Quality of mRNA Vaccines and Therapeutics (Draft Guidelines: 3rd Edition)”, 2 Aug. 2024, US Pharmacopeia Expert Committee: Biologics Monograph 3—Complex Biologics & Vaccines. https://www.uspnf.com/notices/analytical-procedures-mrna-vaccines-20240802. As these procedures require costly equipment, specialized laboratories and expertise, they remain inaccessible to most basic and preclinical researchers. To improve quality control, more effective, efficient, and accessible methods are needed to quantitate the capped RNA content. This invention allows for determination of capping efficiency using simple electrophoretic separation methods, including standard urea denaturing polyacrylamide gel electrophoresis (PAGE). Only a minority of published basic research studies describing production of novel mRNAs evaluated scientifically describe assessment of mRNA quality attributes. Often these evaluations involve the use of agarose or polyacrylamide gels or automated electrophoresis instruments to evaluate the approximate molecular weight and/or purity of full-length mRNAs, and these studies do not permit determination of mRNA capping efficiency. In this regard, see McKenzie R E et al. “mRNA Synthesis and Encapsulation in Ionizable Lipid Nanoparticles”, Curr Protoc. 2023 September; 3(9):e898. doi: 10.1002/cpz1.898.

There is an urgent need for determination of 5′ capping efficiency of mRNAs with standard laboratory equipment and with accuracy comparable to more complicated procedures that make use of costly equipment.

The present invention solves said problem and urgent need by the compositions, kits and methods as outlined below and as defined in the appended claims, allowing for the determination of capping efficiency (percent capped RNA content) of an mRNA sample that has been produced with standard laboratory equipment and with accuracy comparable to more complicated procedures making use of costly equipment and is more broadly applicable to preclinical and basic research workflows. 5′-cap structures can be added co-transcriptionally or post-transcriptionally to in vitro-transcribed (IVT) RNA, producing Cap 0 and/or Cap 1 structures as a step in the mRNA synthesis process. As noted above, since only capped mRNAs will be efficiently translated in cells and uncapped RNAs present may induce an innate immune response due to their resemblance to viral RNAs, it is crucially important to evaluate the capping efficiency following the process of mRNA production.

As a background for this application, the following processes may be applied for making mRNA for therapeutic applications: A first process in making mRNA is in vitro transcription (IVT) of a linear dsDNA template encoding any desired mRNA (whether naturally occurring, or a product of genetic engineering) using any suitable RNA polymerase (e.g., T7, SP6 or T3 RNA polymerase, preferably a T7 RNA polymerase) to generate in vitro-transcribed RNA that has a 5′ triphosphate. The order and type of our other processes for making mRNA can vary, depending on choices of options described in the following items. If the DNA template was genetically engineered to include a polydeoxythymidine (poly(dT)) at the end of the template strand encoding the mRNA, then the RNA polymerase used for IVT would also add a template-encoded poly(A) tail at the 3′ end of the IVT-RNA co-transcriptionally, which is one option for the second process of making mRNA. However, since it has been observed that poly(dT) sequences in plasmids that are maintained and replicated in E. coli host cells and that encode poly(A) tails that are longer than about 50 A's are not stable and that portion of the plasmid mutates to become shorter and to encode variable poly(A) tail lengths, another option for the second process for making mRNA is usually to add the poly(A) tail to the IVT-RNA post-transcriptionally using a poly(A) polymerase enzyme. Adding the poly(A) tail using poly(A) polymerase enzyme in vitro enables production of poly(A) tails comprising at least 150, 175, 200, 250, 300 or >300 Adenine bases, which can result in higher protein expression and greater stability of many mRNAs. A third process for making mRNA is post-transcriptionally 5′-capping IVT-RNA that has been post-transcriptionally 3′-polyadenylated using a capping enzyme system (e.g., a Vaccinia virus capping enzyme system). However, 5′-capping can also be performed co-transcriptionally by including a cap analog such as an “anti-reverse cap analog” (“ARCA”) or other types of cap analogs, which provides the option for post-transcriptional 3′-polyadenylation with a poly(A) polymerase being performed after IVT and co-transcriptional capping.

### Definitions

“RNA” shall be interpreted as any polymer containing multiple ribonucleic acids able to perform the function of an RNA in vivo or in vitro.

“Polynucleotide” shall be interpreted as a biopolymer composed of nucleotide molecules covalently bound in a chain and may be composed of RNA bases (natural and/or man-made), DNA bases (natural and/or man-made), or a combination thereof.

“Nuclease” shall be interpreted as any enzyme or catalyst able to break a polynucleotide up into smaller parts.

“Analog” shall be interpreted as any chemical compound derived from an identified chemical through structural modification.

### DESCRIPTION OF THE INVENTION

The invention described and claimed herein has may attributes and embodiments including, but not limited to, those set forth or described or referenced in this section of the patent application, to be all-inclusive, and the invention described and claimed herein is not limited to or by the features or embodiments, identified in this introduction, which is included for purposes of illustration only and not restriction. This invention includes an oligonucleotide probe that directs a nuclease to a precise location proximal to the 5′ terminus of mRNAs to allow generation of precisely defined RNA fragments that are either 5′ capped or not. For the sake of completeness, it should be noted that the invention is applicable to m7GTP caps and also cap analogs, including but not limited to anti-reverse cap analog (ARCA), unlocked nucleic acid analogs, and modified or unmodified dinucleotide, trinucleotide, and tetranucleotide or larger cap analogs, including any such cap analogs that have been modified as described in WIPO PCT patent application WO2007120863A2, which is incorporated herein by reference in its entirety.

This invention includes different embodiments of oligonucleotide probes that, when hybridized to a defined location near the 5′-end of mRNA, enables a nuclease to cleave the mRNA at a precise location proximal to the 5′ terminus of mRNAs, thereby generating precisely defined RNA fragments that are either 5′ capped or not. This invention describes methods, kits and compositions for analyzing these fragments via simple electrophoretic separation methods (e.g. polyacrylamide gel electrophoresis), permitting routine determination of 5′ capping efficiency in mRNA samples. This invention permits for determination of 5′ capping efficiency of mRNAs with standard laboratory equipment and with accuracy comparable to more complicated procedures that make use of costly equipment. Prior to going into the details of the present invention, some general aspects underlying the impact and advantages of the present invention are outlined below.

Quality assessment (QA) and quality control (QC) of mRNAs under clinical evaluation or used as therapeutics is evident, however the proper determination of mRNA quality in early development is also crucial for ensuring reproducibility of scientific findings and to allow for proper evaluation of preclinical candidates. This invention permits capping efficiency determination independent of the 5′cap present, whether they be naturally occurring m7GTP caps or cap analogs.

The 5′-caps to be analyzed and of which the capping efficiency is to be quantified can be added to mRNAs co-transcriptionally or post-transcriptionally. The claimed compositions, kits, methods, and uses disclosed herein are particularly useful for quick and easy quality assurance (“QA”) and/or quality control (“QC”) of 5′-caps of in vitro-synthesized RNAs for therapeutic and prophylactic uses in humans, animals, including mammals, and other eukaryotes. Some embodiments include Solid phase Reversible Immobilization (SPRI) purification of an RNA sample prior to analysis of capping efficiency, as exemplarily used and shown versus other methods in FIG. 7. In preferred embodiments, the RNA portion of the targeting oligonucleotide is constituted of unmodified bases. However in some other embodiments, the RNA portions of the targeting oligonucleotide probe comprise or consist of 2′-O-methyl-ribonucleotides to increase their stability/shelf-life in the presence of one or more nucleases (e.g. RNase A).

The invention as defined in the claims is outlined in greater detail below.

In one aspect, the invention provides methods of quantitating the relative amounts or percentages of RNA molecules that are 5′-capped and that are uncapped in an RNA sample or for quantifying the efficiency of capping of an in vitro-synthesized mRNA of interest that has been subjected to a capping enzyme system. Three embodiments of said methods are described below, wherein said methods comprise steps a) to e).

A first embodiment of the methods is the following: In step a), an RNA sample is provided, wherein said sample comprises mRNA with a cap structure at the most 5′-end ribonucleotide of said mRNA of interest and mRNA without said cap structure at the most 5′-end ribonucleotide of said mRNA of interest. In step b), said RNA sample is contacted with an 5′RNA-DNA-RNA3′ targeting oligonucleotide consisting of a DNA central portion flanked at its 5′-end and its 3′-end by RNA portions, under conditions that permit said targeting oligonucleotide, including said DNA central portion, to hybridize to said mRNA of interest to form a hybrid between the mRNA of interest and the targeting oligonucleotide, wherein said targeting oligonucleotide is further defined as follows: said DNA central portion comprises or consists of four to six deoxyribonucleotides in length, wherein said DNA central portion is complementary to a sequence at the 5′-end portion of said mRNA of interest within 15 to 50 bases, preferably within 24 to 40 bases from said most 5′ end ribonucleotide, each of said RNA flanking portions comprises 5 to 15, preferably 8-12, further preferably 10 ribonucleotides in length and said RNA flanking portions are complementary to corresponding RNA portions in said mRNA of interest flanking the sequence hybridized to the targeting oligonucleotide's DNA central portion. In step c), a nuclease is provided that selectively cleaves RNA in a DNA/RNA hybrid, resulting in the cleavage of said mRNA of interest in the sequence region hybridized to the targeting oligonucleotide's DNA central portion and the release of the cleaved mRNA 5′-end portion fragments with said cap structure and the cleaved mRNA 5′-end portion fragments without said cap structure. In step d), said released mRNA 5′-end portion fragments are electrophoretically separated, preferably by gel electrophoresis in a polyacrylamide electrophoresis gel, or using an electrophoretic separation instrument or device comprising a gel or a capillary tube, a column containing a gel or a sizing matrix, or a cartridge containing a gel or a sizing matrix for a bioanalyzer fragment analyzer, or capillary electrophoresis instrument. In step e), the relative amount of said separated capped fragments and uncapped fragments is determined, thereby quantifying the efficiency of mRNA capping in said mRNA of interest.

A second embodiment of the methods is the following: In step a), an RNA sample is provided, wherein said sample comprises mRNA with a cap structure at the most 5′-end ribonucleotide of said mRNA of interest and mRNA without said cap structure at the most 5′-end ribonucleotide of said mRNA of interest. In step b), said mRNA sample is contacted with an 5′DNA-RNA3′ targeting oligonucleotide consisting of a 5′ DNA portion and a 3′ RNA portion, under conditions that permit said targeting oligonucleotide, including said DNA portion, to hybridize to said mRNA of interest to form hybrid between an mRNA of interest and a targeting oligonucleotide, wherein said targeting oligonucleotide is further defined as follows: said DNA portion comprises or consists of four to six deoxyribonucleotides in length, wherein said DNA portion is complementary to a sequence at the 5′-end portion of said mRNA of interest within 15 to 50 bases, preferably within 24 to 40 bases from said most 5′ end ribonucleotide, said RNA portion comprises 5 to 15, preferably 8-12, further preferably 10 ribonucleotides in length and said RNA portion is complementary to a corresponding RNA portion in said mRNA of interest flanking the sequence hybridized to the targeting oligonucleotide's DNA portion. In step c), a nuclease is provided that selectively cleaves RNA in a DNA/RNA hybrid, resulting in the cleavage of said mRNA of interest in the sequence region hybridized to the targeting oligonucleotide's DNA portion and the release of the cleaved mRNA 5′-end portion fragments with said cap structure and the cleaved mRNA 5′-end portion fragments without said cap structure. In step d), said released mRNA 5′-end portion fragments are electrophoretically separated, preferably by gel electrophoresis in a polyacrylamide electrophoresis gel, or using an electrophoretic separation instrument or device comprising a gel or a capillary tube, a column containing a gel or a sizing matrix, or a cartridge containing a gel or a sizing matrix for a bioanalyzer fragment analyzer, or capillary electrophoresis instrument. In step e), the relative amount of said separated capped fragments and uncapped fragments is determined, thereby quantifying the efficiency of mRNA capping in said mRNA of interest.

A third embodiment of the methods is the following: In step a), an RNA sample is provided, wherein said sample comprises mRNA with a cap structure at the most 5′-end ribonucleotide of said mRNA of interest and mRNA without said cap structure at the most 5′-end ribonucleotide of said mRNA of interest. In step b), said mRNA sample is contacted with a 5′DNA-RNA3′ targeting oligonucleotide consisting of a 5′DNA portion and a 3′ RNA portion, under conditions that permit said targeting oligonucleotide, including said DNA portion, to hybridize to said mRNA of interest to form a hybrid between the mRNA of interest and the targeting oligonucleotide, wherein said targeting oligonucleotide is further defined as follows: said DNA portion comprises or consists of four to six deoxyribonucleotides in length, wherein said DNA portion is complementary to a sequence at the 5′-end portion of said mRNA of interest within 15 to 50 bases, preferably within 24 to 40 bases from said most 5′ end ribonucleotide, said RNA portion comprises 5 to 15, preferably 8-12, further preferably 10 ribonucleotides in length and said RNA portion is complementary to a corresponding RNA portion in said mRNA of interest flanking the sequence hybridized to the targeting oligonucleotide's DNA portion, said targeting oligonucleotide further comprises downstream and adjacent to the 3′ end of the DNA portion at least one ribonucleotide, wherein said at least one ribonucleotide is complementary to the counterpart ribonucleotide of the mRNA of interest. In step c), a nuclease is provided that selectively cleaves RNA in a DNA/RNA hybrid, resulting in the cleavage of said mRNA of interest in the sequence region hybridized to the targeting oligonucleotide's DNA portion and the release of the cleaved mRNA 5′-end portion fragments with said cap structure and the cleaved mRNA 5′-end portion fragments without said cap structure. In step d), said released mRNA 5′-end portion fragments are electrophoretically separated, preferably by gel electrophoresis in a polyacrylamide electrophoresis gel, or using an electrophoretic separation instrument or device comprising a gel or a capillary tube, a column containing a gel or a sizing matrix, or a cartridge containing a gel or a sizing matrix for a bioanalyzer fragment analyzer, or capillary electrophoresis instrument. In step e), the relative amount of said separated capped fragments and uncapped fragments are determined, thereby quantifying the efficiency of mRNA capping in said mRNA of interest.

In preferred embodiments of the methods, said mRNA of interest is produced by in vitro transcription (IVT) of a DNA template encoding said mRNA of interest, and wherein said cap structure is post- or co-transcriptionally added to the most 5′-end ribonucleotide of said mRNA of interest. In further preferred embodiments of said methods, said IVT produced mRNA with said cap structure comprises an additional N7-methylated guanosine linked by a 5′ to 5′ triphosphate bridge to the 5′-end of said IVT mRNA as shown below

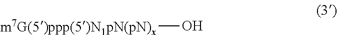

wherein m⁷G represents the N7-methylguanosine cap nucleoside, ppp represents the triphosphate bridge between the 5′ carbons of the cap nucleoside and the first ribonucleotide of the primary RNA transcript, and N₁pN(pN)ₓ—OH (3′) represents the primary RNA transcript, of which N₁ is the most 5′-end ribonucleotide.

In preferred embodiments, said nuclease only cleaves RNA that is hybridized to DNA and said nuclease has a minimum binding domain size of four nucleotides, wherein said nuclease is preferably RNase H. RNase H is known to specifically cleave RNA hybridized with DNA in the region of the hybridization and efforts to focus RNase H to a unique cleavage site include use of complementary 2′-O-methyl oligonucleotides containing a tetradeoxyribonucleotide. In this regard, see Inoue et al., “Sequence-dependent hydrolysis of RNA using modified oligonucleotide splints and RNase H”, FEBS Lett. 1987 May 11; 215(2):327-30. doi: 10.1016/0014-5793(87)80171-0; Lapham, J and Crothers, D M, 1996, “RNase H cleavage for processing of in vitro transcribed RNA for NMR studies and RNA ligation”, Rna, 2(3), 289-296; and Lapham et al., “The position of site-directed cleavage of RNA using RNase H and 2′-O-methyl oligonucleotides is dependent on the enzyme source”, RNA, 1997 September; 3(9):950-1”. Patented methods describe making use of RNase H in combination with DNA probes prior to analyzing degree of mRNA capping by chromatography. In this regard, see U.S. Pat. No. 11,365,437B2. Combining probe-directed RNase H cleavage with analysis by polyacrylamide gels has also been described as a simple and low-cost method requiring many handling steps and 5 hours of sample processing time. In this regard, see Tu et al., 2024, “Capped or uncapped? Techniques to assess the quality of mRNA molecules”, Current Opinion in Systems Biology, 100503. Other embodiments include the use of an alternative RNase, e.g. RNase 4 in place of RNase H.

In preferred embodiments, said nuclease cleaves said mRNA of interest only once.

In preferred embodiments, said gel is a polyacrylamide gel, preferably a polyacrylamide gel comprising 17% to 22% acrylamide, further preferably comprising 20% acrylamide, including 19.5% acrylamide and 0.50% Bis-acrylamide.

In preferred embodiments, said gel is 20% acrylamide/8 M Urea/1×TBE gel.

In preferred embodiments, said released mRNA 5′-end portion fragments are fragments are larger than the targeting oligo, and, more preferably, said released mRNA 5′-end portion fragments are fragments of 25 to 40 nucleotides in length.

In preferred embodiments, said targeting oligonucleotide is further defined by one of the following features or any combination(s) thereof. i) the Tₘ of said targeting oligonucleotide (7) is 40° C. to 60° C., and, more preferably, the Tₘ of said targeting oligonucleotide (7) is about 50° C.; ii) the targeting oligo (TO) anneals to an mRNA having an autologous 5′-untranslated region (5′ UTR); iii) the targeting oligo (TO) anneals to a heterologous 5′-untranslated region (5′ UTR) that is used for many different mRNAs, enabling use of the same targeting oligo for assaying the 5′ cap status of any mRNA that has it; iv) the targeting oligo anneals to a Xenopus 5′ UTR, preferably, the targeting oligo (TO) anneals to a Xenopus alpha-globin or beta-globin 5′-UTR; v) the targeting oligo anneals to a Xenopus 5′ UTR, preferably, the targeting oligo (TO) anneals to a human alpha-globin or beta-globin 5′-UTR; vi) said targeting oligo is designed to so the DNA portion does not hybridize to counterpart ribonucleotides in sequence at the 5′-end portion of the mRNA of interest that comprise modified ribonucleosides selected from pseudouridine and N1-methylpseudouridine; vii) the length of the targeting oligonucleotide in terms of nucleotides is different from the released mRNA 5′ end portion fragments, wherein the length of released mRNA 5′ end portion fragments, is preferably 5 to 20 nucleotides longer than the targeting oligonucleotide.

In some preferred embodiments, no marker fragments are used to determine the lengths of the released mRNA 5′-end portion fragments or the targeting oligo. In some preferred embodiments, the methods further comprise the step of providing i) a collection of molecular weight markers, including a marker ladder, preferably a marker ladder comprising or consisting of a collection of RNA and/or single-stranded DNA marker fragments of different lengths, wherein the length of said marker fragments, in terms of nucleotides, is preferably a multiple of an integer of 10, wherein further preferably the shortest marker fragment has a length of 10 nucleotides and the longest marker fragment has a length of 60 nucleotides; and/or ii) a control mixture of 5′-end capped and uncapped control mRNA fragments, preferably a mixture of control mRNA fragments comprising a ratio in the range of 9:1 to 1:9, including 3:2, 3:1, 1:1, 2:3, 1:3, of capped to uncapped fragments of the same mRNA fragment. In further preferred embodiments, said methods further comprise i) parallel electrophoresing said released mRNA 5′-end portion fragments and said collection of molecular weight markers and/or said control mixture of 5′end capped and uncapped control mRNA fragments on said electrophoresis gel in step d), and ii) comparing the length of the gel separated mRNA 5′-end portion fragments with said weight markers subsequently.

The invention further provides a kit or kit-of-parts for use in the methods as described above, said kit or kit-of-parts comprising a) a nuclease that selectively cleaves RNA in a DNA/RNA hybrid, preferably RNase H, and b) a stop/loading buffer containing at least one dye, and preferably two dyes, further preferably bromophenol blue and xylene cyanol, as electrophoresis marker dyes.

In some embodiments, said kit or kit-of-parts may further comprise an electrophoresis gel, wherein said electrophoresis gel is preferably a polyacrylamide gel, preferably a polyacrylamide gel comprising 17% to 22% acrylamide, further preferably comprising 20% acrylamide, including 19.5% acrylamide and 0.50% Bis-acrylamide.

In some embodiments, said kit or kit or kit-of-parts may further comprise a collection of molecular weight markers, including a marker ladder, preferably a marker ladder comprising or consisting of a collection of RNA and/or single-stranded DNA marker fragments of different lengths, wherein the length of said marker fragments, in terms of nucleotides, is preferably a multiple of an integer of 10, wherein further preferably the shortest marker fragment has a length of 10 nucleotides and the longest marker fragment has a length of 60 nucleotides.

In some embodiments, the kit or kit-of-parts may further comprise an RNA staining dye, wherein said RNA staining dye preferably comprises ethidium bromide, GelRed™ and/or oxazole gold or SYBRGold.

The invention further provides the use of a nuclease that selectively cleaves RNA in a DNA/RNA hybrid in a method as described above, wherein, preferably, said nuclease is RNase H.

### DESCRIPTION OF THE DRAWINGS AND REFERENCE SIGNS

The following reference signs are used for the corresponding features in FIGS. 1, 2 and 3

| Reference | |
|---|---|
| sign | Feature |
| 1 | mRNA of interest |
| 1a | mRNA with a cap structure 2 at the most 5′-end ribonucleotide 3 of the |
| | mRNA of interest 1 |
| 1b | mRNA without a cap structure 2 at the most 5′-end ribonucleotide 3 of |
| | the mRNA of interest 1 |
| 2 | Cap structure |
| 3 | most 5′-end ribonucleotide of the mRNA of interest 1 |
| 4 | Sequence at the 5′-end of the mRNA of interest 1 within 15 to 50 bases |
| | from the most 5′-end ribonucleotide 3 |
| 5, 6 | RNA portions flanking sequence 4 on both sides |
| 7 | Targeting oligonucleotide |
| 8 | DNA central portion of the targeting oligonucleotide 7 comprising or |
| | consisting of four to six deoxyribonucleotides in length |
| 9, 10 | RNA portions flanking the DNA central portion 8 of the targeting |
| | nucleotide 7 on both sides and comprising 5 to 15 ribonucleotides in |
| | length, and complementary to the corresponding RNA portions 5 and 6 |
| | in the mRNA of interest 1 |
| 11 | nuclease that selectively cleaves RNA in a DNA/RNA hybrid |
| 12a | cleaved mRNA 5′-end portion fragment with cap structure 2 |
| 12b | cleaved mRNA 5′-end portion fragment without cap structure 2 |

FIG. 1 shows the first embodiment of the methods according to claim 1 as filed.

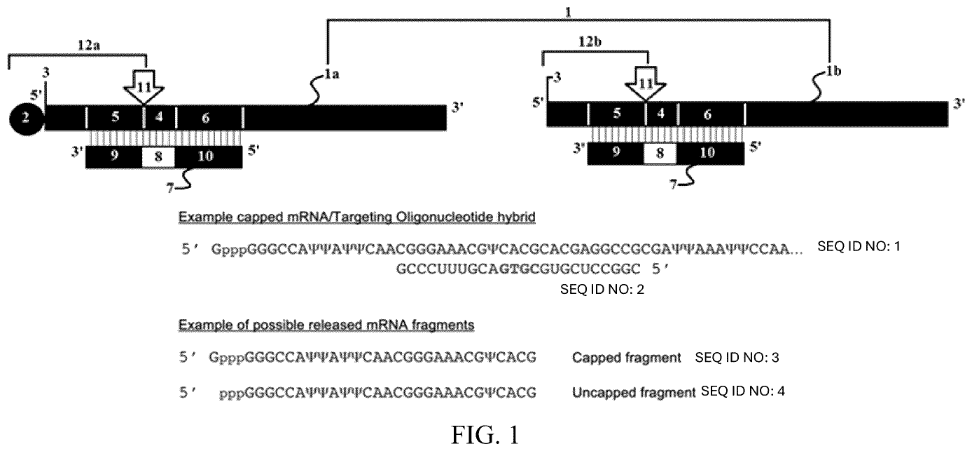

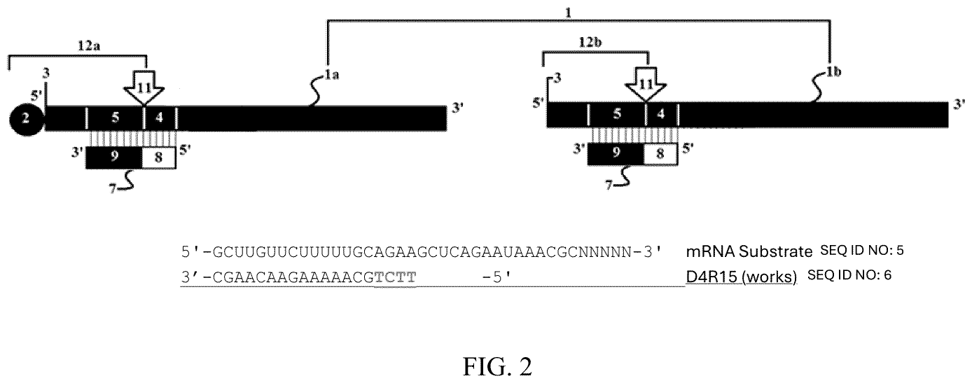

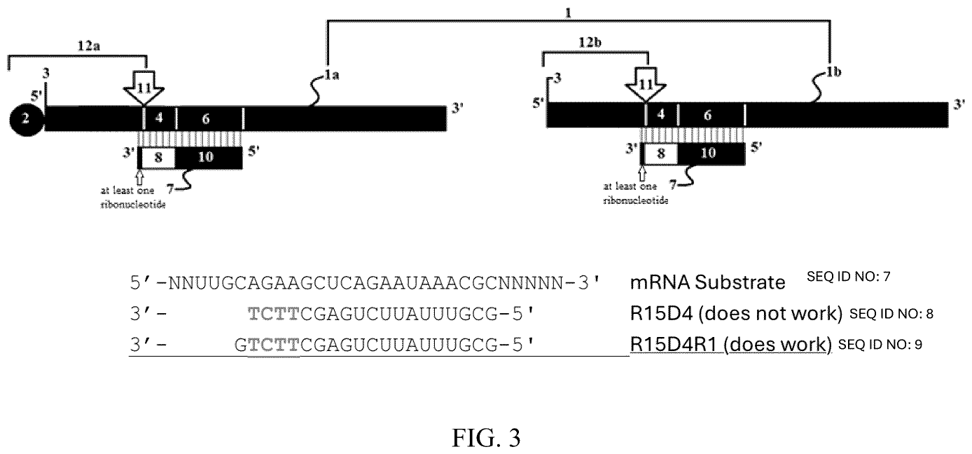

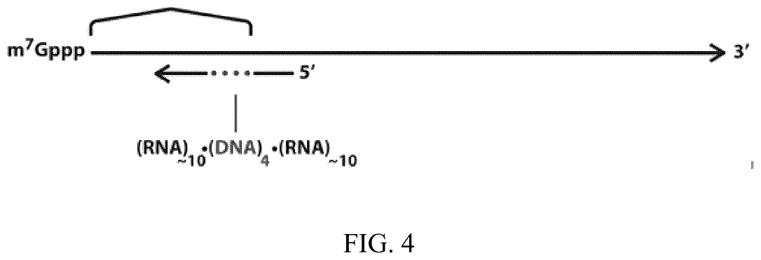

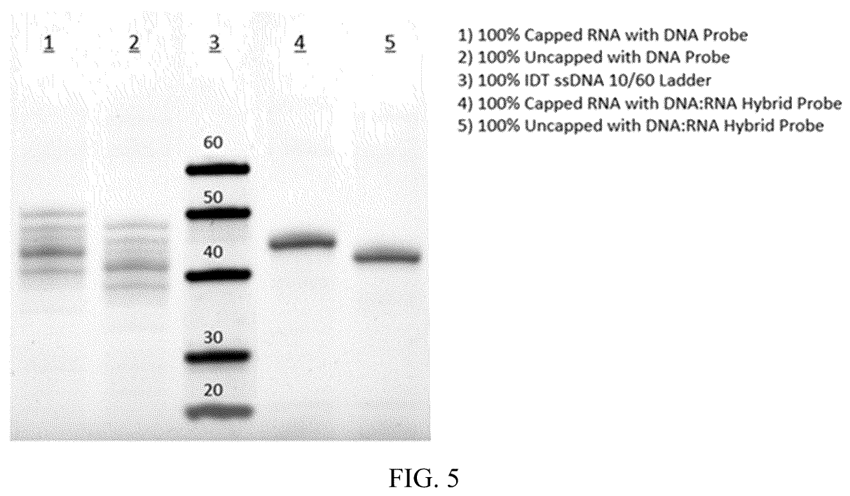

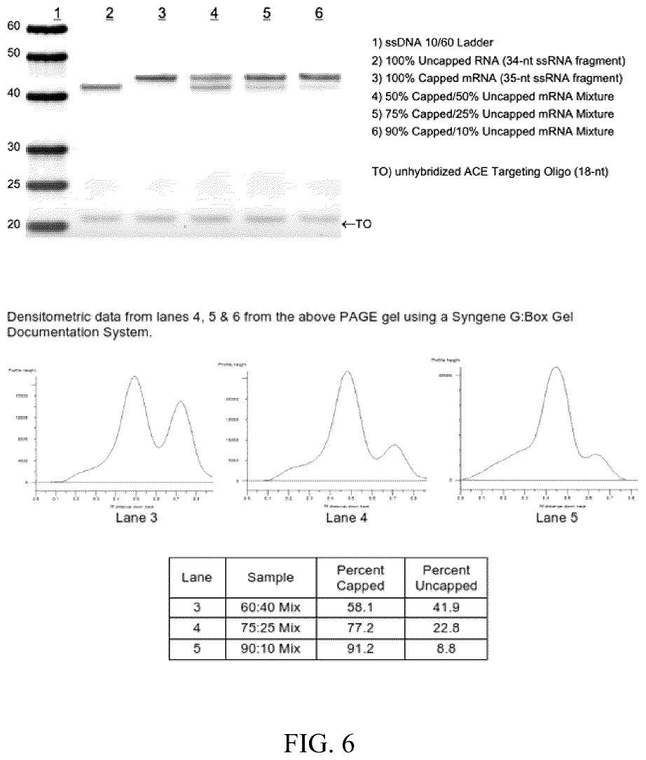

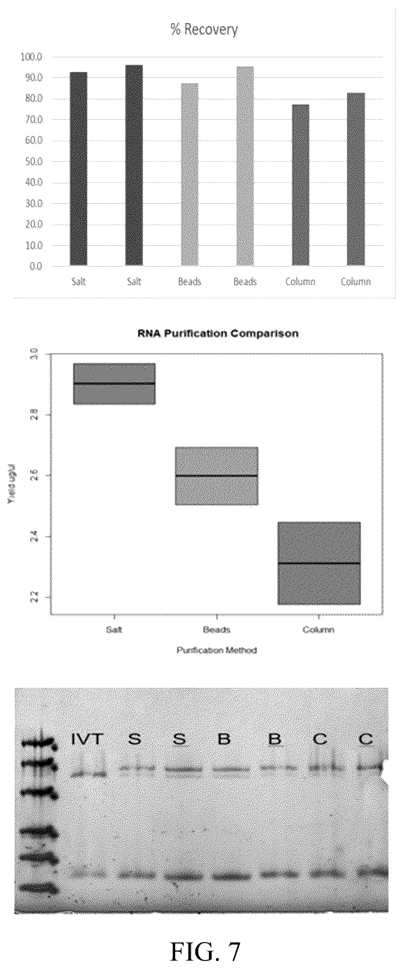

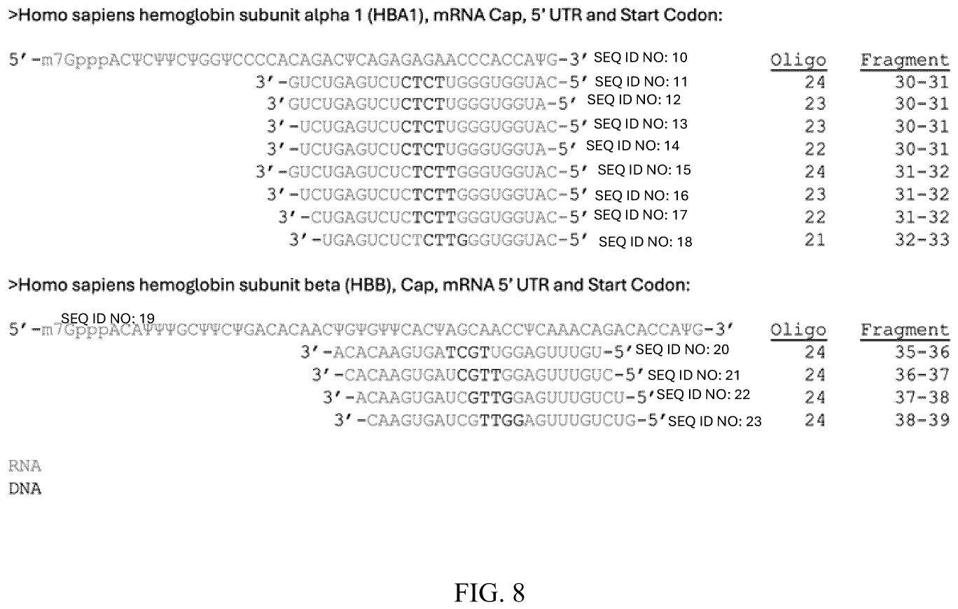

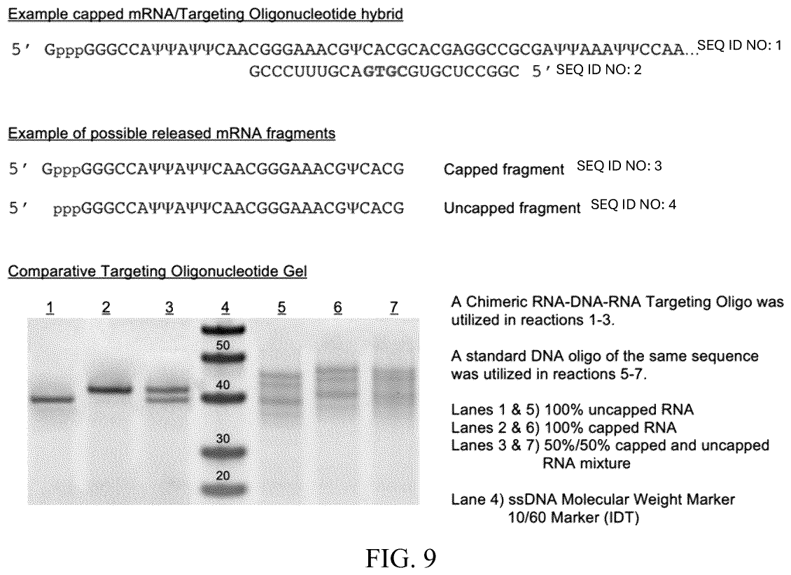

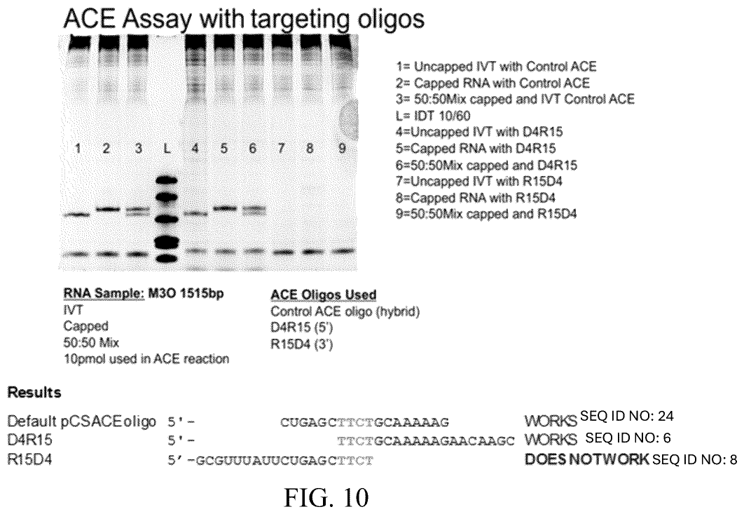

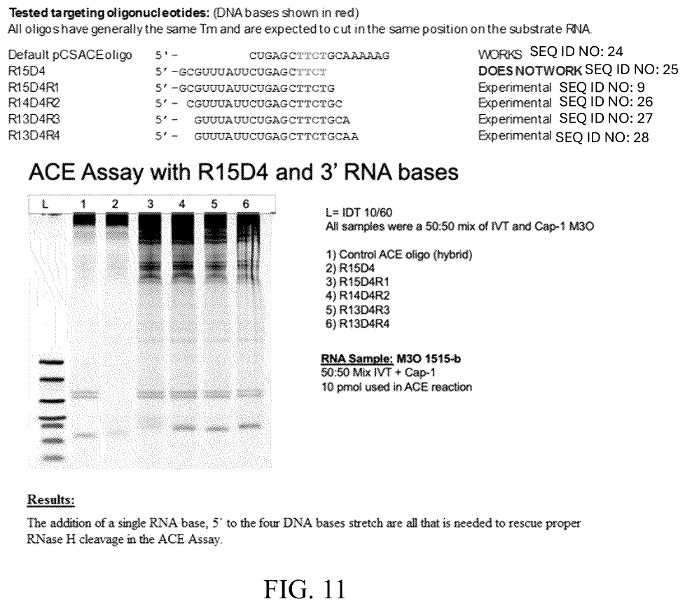

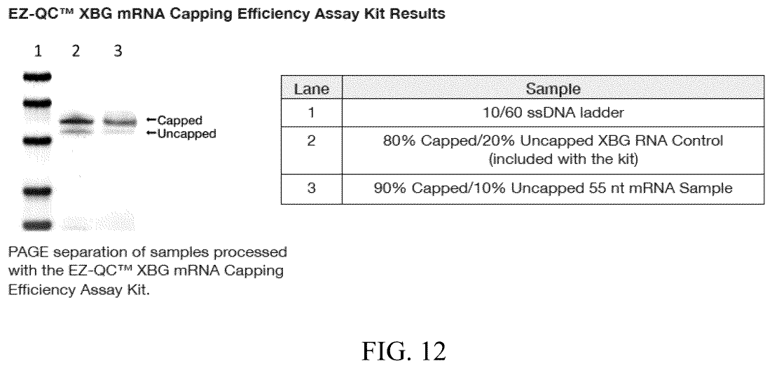

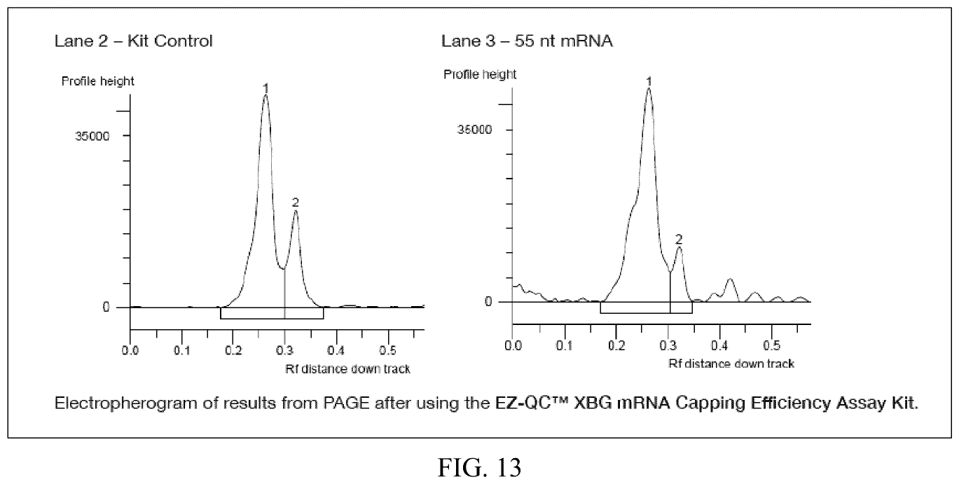

An RNA sample is provided, wherein said sample comprises mRNA 1a with a cap structure 2 at the most 5′-end ribonucleotide 3 of said mRNA of interest 1 and mRNA 1b without said cap structure 2 at the most 5′-end ribonucleotide 3 of said mRNA of interest 1. Said RNA sample is contacted with an 5′RNA-DNA-RNA3′ targeting oligonucleotide 7 consisting of a DNA central portion 8 flanked at its 5′-end and its 3′-end by RNA portions 9, 10. Said targeting oligonucleotide 7, including said DNA central portion 8, is hybridized to said mRNA of interest 1 to form a hybrid between the mRNA of interest 1 and the targeting oligonucleotide 7. Said targeting oligonucleotide 7 comprises said DNA central portion 8 comprising or consisting of four to six deoxyribonucleotides in length, wherein said DNA central portion 8 is complementary to a sequence 4 at the 5′-end portion of said mRNA of interest 1 within 15 to 50 bases, preferably within 24 to 40 bases from said most 5′ end ribonucleotide 3. Each of said RNA flanking portions 9, 10 comprises 5 to 15, preferably 8-12, further preferably 10 ribonucleotides in length and said RNA flanking portions 9, 10 are complementary to corresponding RNA portions 5, 6 in said mRNA of interest 1 flanking the sequence 4 hybridized to the targeting oligonucleotide's DNA central portion 8. A nuclease 11 selectively cleaves RNA in a DNA/RNA hybrid, resulting in the cleavage of said mRNA of interest 1 in the sequence region hybridized to the targeting oligonucleotide's DNA central portion 8 and the release of the cleaved mRNA 5′-end portion fragments 12a with said cap structure 2 and the cleaved mRNA 5′-end portion fragments 12b without said cap structure 2. Said released mRNA 5′-end portion fragments 12a, 12b may be electrophoretically separated, preferably by gel electrophoresis in a polyacrylamide electrophoresis gel, or using an electrophoretic separation instrument or device comprising a gel or a capillary tube, a column containing a gel or a sizing matrix, or a cartridge containing a gel or a sizing matrix for a bioanalyzer fragment analyzer, or capillary electrophoresis instrument and then the relative amount of said separated capped fragments 12a and uncapped fragments 12b may be determined, thereby quantifying the efficiency of mRNA capping in said mRNA of interest 1. The latter electrophoresing and determining steps are not shown in FIG. 1.

FIG. 2 shows one embodiment of the method of claim 2 as filed.

An RNA sample is provided, wherein said sample comprises mRNA 1a with a cap structure 2 at the most 5′-end ribonucleotide 3 of said mRNA of interest 1 and mRNA 1b without said cap structure 2 at the most 5′-end ribonucleotide 3 of said mRNA of interest 1. Said mRNA sample is contacted with a 5′DNA-RNA3′ targeting oligonucleotide 7 consisting of a 5′ DNA portion 9 and a 3′ RNA portion 8. Said targeting oligonucleotide 7, including said DNA portion 8, is hybridized to said mRNA of interest 1 to form hybrid between an mRNA of interest 1 and the targeting oligonucleotide 7. Said DNA portion 8 of said targeting oligonucleotide 7 comprises or consists of four to six deoxyribonucleotides in length, wherein said DNA portion 8 is complementary to a sequence 4 at the 5′-end portion of said mRNA of interest 1 within 15 to 50 bases, preferably within 24 to 40 bases from said most 5′ end ribonucleotide 3. Said RNA portion 9 comprises 5 to 15, preferably 8-12, further preferably 10 ribonucleotides in length and said RNA portion 9 is complementary to a corresponding RNA portion 5 in said mRNA of interest 1 flanking the sequence 4 hybridized to the targeting oligonucleotide's DNA portion 8. A nuclease 11 selectively cleaves RNA in a DNA/RNA hybrid, resulting in the cleavage of said mRNA of interest 1 in the sequence region hybridized to the targeting oligonucleotide's DNA portion 8 and the release of the cleaved mRNA 5′-end portion fragments 12a with said cap structure 2 and the cleaved mRNA 5′-end portion fragments 12b without said cap structure 2. Said released mRNA 5′-end portion fragments 12a, 12b may be electrophoretically separated, preferably by gel electrophoresis in a polyacrylamide electrophoresis gel, or using an electrophoretic separation instrument or device comprising a gel or a capillary tube, a column containing a gel or a sizing matrix, or a cartridge containing a gel or a sizing matrix for a bioanalyzer fragment analyzer, or capillary electrophoresis instrument and then the relative amount of said separated capped fragments 12a and uncapped fragments 12b may be determined, thereby quantifying the efficiency of mRNA capping in said mRNA of interest 1. The latter electrophoresing and determining steps are not shown in FIG. 2.

FIG. 3 shows one embodiment of the method of claim 3 as filed.

An RNA sample is provided, wherein said sample comprises mRNA 1a with a cap structure 2 at the most 5′-end ribonucleotide 3 of said mRNA of interest 1 and mRNA 1b without said cap structure 2 at the most 5′-end ribonucleotide 3 of said mRNA of interest 1. Said mRNA sample is contacted with a 5′RNA-DNA3′ targeting oligonucleotide having at least one ribonucleotide 7 consisting of a 3′ DNA portion 8 and a 5′ RNA portion 10. Said targeting oligonucleotide having at least one ribonucleotide 7, including said DNA portion 8, is hybridized to said mRNA of interest 1 to form a hybrid between the mRNA of interest 1 and the targeting oligonucleotide having at least one ribonucleotide 7. Said DNA portion 8 of said 5′RNA-DNA3′ targeting oligonucleotide having at least one ribonucleotide 7 comprises or consists of four to six deoxyribonucleotides in length, wherein said DNA portion and said at least one ribonucleotide 8 is complementary to a sequence 4 at the 5′-end portion of said mRNA of interest 1 within 15 to 50 bases, preferably within 24 to 40 bases from said most 5′ end ribonucleotide 3. Said RNA portion 10 comprises 5 to 15, preferably 8-12, further preferably 10 ribonucleotides in length and said RNA portion 10 is complementary to a corresponding RNA portion 6 in said mRNA of interest 1 flanking the sequence 4 hybridized to the targeting oligonucleotide's DNA portion 8. Said targeting oligonucleotide 7 further comprises downstream and adjacent to the 3′ end of the DNA portion 8 at least one ribonucleotide, wherein said at least one ribonucleotide is complementary to the counterpart ribonucleotide of the mRNA of interest 1. A nuclease 11 selectively cleaves RNA in a DNA/RNA hybrid, resulting in the cleavage of said mRNA of interest 1 in the sequence region hybridized to the targeting oligonucleotide's DNA portion 8 and the release of the cleaved mRNA 5′-end portion fragments 12a with said cap structure 2 and the cleaved mRNA 5′-end portion fragments 12b without said cap structure 2. Said released mRNA 5′-end portion fragments 12a, 12b may be electrophoretically separated, preferably by gel electrophoresis in a polyacrylamide electrophoresis gel, or using an electrophoretic separation instrument or device comprising a gel or a capillary tube, a column containing a gel or a sizing matrix, or a cartridge containing a gel or a sizing matrix for a bioanalyzer fragment analyzer, or capillary electrophoresis instrument and then the relative amount of said separated capped fragments 12a and uncapped fragments 12b may be determined, thereby quantifying the efficiency of mRNA capping in said mRNA of interest 1. The latter electrophoresing and determining steps are not shown in FIG. 3.

FIG. 4 is a drawing depicting the hybridization of an RNA:DNA:RNA chimeric oligonucleotide to an mRNA molecule such that the most 5′ DNA base hybridizes 25-50 nucleotides from the 5′ cap.

FIG. 5 shows digestion of capped and uncapped mRNA samples by RNase H guided by RNA:DNA:RNA chimeric oligos compared with a DNA probes. While use of DNA probes leads to cutting of mRNA at multiple locations yielding 6+ bands for each sample, a RNA:DNA:RNA probe yields single, distinct bands for capped and uncapped mRNAs optimal for quantitative analysis by polyacrylamide gel electrophoresis. Capped (lanes 1 & 4) and uncapped (lanes 2 & 5) RNAs were tested with both a DNA oligonucleotide and an RNA:DNA:RNA chimeric oligonucleotide. The DNA oligo results in a multiple banding pattern as a result of RNase H cleavage at multiple locations along the length of the oligo/mRNA hybridization. The RNA:DNA:RNA chimeric oligo results in a specific band due to the chimeric oligo directing the RNase H cleavage to a single specific locus in the oligo/mRNA hybridization length. This increased specificity allows for greater confidence in determining the percentage capped/uncapped content of the assayed mRNA sample.

FIG. 6 shows example data with mixed capped and uncapped mRNA samples. Percent of capped vs. noncapped mRNA digested by RNase H guided by RNA:DNA:RNA probes is visible and measurable using 20% acrylamide, 8 M Urea, 1×TBE gel stained with SYBR-Gold (5 ul per 50 ml water). Upper:Lower band ratios correlate strongly with known values.

FIG. 7 shows post capping purification with SPRI (Solid phase Reversible Immobilization) prior to analysis by polyacrylamide gel electrophoresis yields comparable results to salt-based or column purification. As can also be seen, SPRI beads are comparable to salt purification, but columns resulted in slightly lower yields.

FIG. 8 shows examples of RNA-DNA-RNA probes to the 5′UTR (alpha-globin and beta-globin) and start codon of capped, nucleoside-substituted mRNAs. Constraints for: a) the probe to be distinct in apparent molecular weight from the cleaved cap, b) the DNA portion to be at least 4 nucleotides long, c) the RNA arms to be approximately 10 bases, d) the RNA arms to be approximately equal in length, e) the DNA portion not to hybridize with the modified nucleoside, f) and the size of the cleaved cap to be less than 50 nts in length limit the choice of probes to be used.

FIG. 9 shows an example targeting oligo design and demonstrated superiority of RNA-DNA-RNA oligo to DNA oligo alone. For the shown mRNA 5′ sequence an oligonucleotide probe is shown (upper). Based on the design the capped or uncapped mRNA fragments will be generated by RNase H when an RNA-DNA-RNA probe is used (middle). Polyacrylamide gel electrophoresis reveals much distinct banding representing capped and uncapped mRNA samples when the RNA-DNA-RNA targeting oligo is used (lower).

FIG. 10 and FIG. 11 refer to the embodiments of claims 2 and 3. As shown in FIG. 10, the 5′DNA-RNA3′ targeting oligonucleotide “D4R15” corresponding to the embodiment of claim 2 worked, while the targeting oligonucleotide “R15D4” having its RNA portion at the DNA portions 5′ end of the targeting oligonucleotide did not work. However, as shown by the data presented in FIG. 11, if at least one ribonucleotide is added at the DNA portion's 3′ end (which corresponds to the embodiment of claim 3), then it works. In other words, in the context of the embodiment of claim 3, the used nuclease (RNase H) needs one or more RNA bases downstream of the last of the four DNA bases to function in the assay.

FIG. 12 shows PAGE analysis of capped and uncapped RNA samples using the EZ-QC™ XBG mRNA capping efficiency assay, and FIG. 13 shows the LC-MS characterization of a capped and uncapped RNA mixture, as described in greater detail in Example 5.

### EXAMPLES

The examples included in this description are not intended to limit the claimed invention but are provided solely to illustrate and confirm the achievement of the expected technical results. These examples are among many experimental data obtained by the inventors, which confirm the efficacy of the compositions within the scope of the invention.

### Example 1

Analysis of Percent Capped mRNA in Samples of mRNAs with Varying Degrees of Capping

As shown in FIG. 6, the ratio of capped to uncapped mRNA in mixtures of capped and uncapped mRNAs of differing ratios can be accurately estimated using polyacrylamide gel electrophoresis.

Reaction components include: RNase H (1 microliter supplied in 50% glycerol, 50 mM Tris-HCl, pH 7.5, 100 mM NaCl, 1 mM dithiothreitol (DTT), 0.1 mM EDTA and 0.1% Triton® X-100), 10× Reaction Buffer (1 microliter supplied in 0.2 M Tris-acetate, pH 7.9, 0.5 M potassium acetate, 0.1 M magnesium acetate and 0.01 M DTT), 5 micromolar targeting oligo, ScriptGuard™ RNase Inhibitor (20 units supplied in 50% glycerol, 50 mM Tris-HCl, pH 7.5, 100 mM NaCl, 10 mM DTT, 0.1 mM EDTA and 0.1% Triton X-100), 5-10 picomoles of the mRNA sample being assayed, and RNase-Free Water (to 10 microliters total). Incubation temperature and time: 37° C. for 30 minutes.

Reaction is stopped by adding 10 microliters of Stop/Loading Buffer (95% formamide, 10 mM EDTA, pH 7.5, 0.01% bromophenol blue and 0.01% xylene cyanol) to each reaction.

Samples are subjected to gel electrophoresis: 20% acrylamide/8 M Urea/I×TBE gel with the gel prepared following these recommendations: a) blow out unpolymerized acrylamide from the wells immediately after comb removal, b) pre-run the gel at 300 volts constant voltage for 20-30 minutes, and c) blow out the excess urea from the wells just prior to sample loading.

Samples should be preheated to 65-70° C. for 5 minutes just prior to gel loading and 5 microliters of each sample should be loaded per lane. Use of known reference standards approximately representing 100% capped and 100% uncapped controls should be loaded next to one another.

Gels should be run at 300 volts (constant voltage) for 2-3 hours until the until the xylene cyanol dye front is approximately 1 cm from the end of the gel; the bromophenol blue dye will have run off the end of the gel.

Gels should be stained with a dye appropriate for your gel visualization system (e.g., SYBR® Gold Nucleic Acid Gel Stain, 5 l per 50 ml water), and the stained gel should be visualized and an image the gel should be captured and relative band intensities quantitated.

### Example 2

### RNA-DNA-RNA Targeting Oligo Design and Superior Performance Compared to DNA Targeting Oligo

As shown in FIG. 9, RNA-DNA-RNA targeting oligonucleotides direct RNase H to cut a defined mRNA sequence uniformly yielding clearly interpretable banding patterns when capped, uncapped or a mixture of the two are assayed. mRNA samples were subjected to the RNase H digestion and gel electrophoresis conditions described in Example 1.

### Example 3

RNA-DNA-RNA Targeting Oligos Functional for Assessing the Capping Efficiency of In Vitro Transcribed mRNA Samples Comprising the 5′ Untranslated Region of Human Alpha Globin or Beta Globin mRNAs Carrying Modified Uridine (Ψ, Including but not Limited to Pseudouridine or N1-Methylpseudouridine)

As shown in FIG. 8, the displayed RNA-DNA-RNA targeting oligos are optimal as they respect the design criteria of a) roughly equal in length RNA arms of approximately 10 bases each, b) four DNA bases designed not to hybridize with the modified uridine bases, c) designed to direct RNase H to cut the 5′ end of the mRNA yielding a fragment of 25-50 bases, but sufficiently distinct in size from the targeting oligo, and d) an oligonucleotide design tool predicts a melting temperature of 50-68° C. for the corresponding DNA oligonucleotide under standard buffer conditions.

### Example 4

### RNA-DNA and DNA-RNA Targeting Oligos Testing Supporting the Embodiments as Defined in the Methods of Claims 2 and 3 as Filed

As shown in FIG. 10, the 5′DNA-RNA3′ targeting oligonucleotide (D4R15) corresponding to the embodiment of claim 2, as filed, worked. The targeting oligonucleotide R15D4 having its RNA portion at the DNA portions 5′ end did not work. However, as shown by the data presented in FIG. 11, if at least one ribonucleotide is added at the DNA portion's end corresponding to the embodiment of claim 3, then it works. In other words, the used nuclease (RNase H) needs one or more RNA bases downstream of the last of the four DNA bases in order to function in the assay.

### Example 5

### LC-MS Benchmarking for Assay of RNA Capping Efficiency

As shown described below and as shown in FIGS. 12-13 in a comparative evaluation, a 55-nucleotide mRNA sample pre-formulated at 90% capped and 10% uncapped RNA was processed using the EZ-QC™ XBG mRNA Capping Efficiency Assay. Said assay uses the EZ-QC™ XBG mRNA Capping Efficiency Assay Kit that provides quantitative capping efficiency (percent capped RNA content) analysis of synthesized mRNA containing a Xenopus beta-globin (XBG) 5′ UTR (untranslated region). Since only capped RNAs are expressed in cells, it is essential to have the highest possible percentage of capped RNAs present in a sample. The EZ-QC™ XBG mRNA Capping Efficiency Assay Kit simplifies analysis and replaces tedious and expensive capping efficiency determination methods such as HPLC and mass spectrometry, instead allowing for determination based upon standard polyacrylamide gel electrophoresis (PAGE) methodology.

The EZ-QC™ XBG mRNA Capping Efficiency Assay utilizes a chimeric RNA-DNA-RNA Targeting Oligonucleotide (kit provided) which hybridizes to the 5′-end region of an RNA mixture when the mRNA construct contains a Xenopus beta-globin 5′ UTR. This hybridization complex serves as a substrate for RNase H cleavage releasing the capped and uncapped 5′-end fragments which can subsequently be resolved via PAGE and quantified by gel band analysis. The method allows for straightforward, fluorescence-based calculation of the percentage capped RNA content of the sample. The specificity of the Targeting Oligonucleotide, designed to facilitate cutting the mRNA at a precise location, provides greater confidence in determining the percentage of capped/uncapped content of the assayed RNA sample. For the sake of completeness, it should be noted that the EZ-QC™ XBG mRNA Capping Efficiency Assay does not differentiate between Cap 0 and Cap 1 capped RNAs, only between capped (Cap 0+Cap 1) and uncapped RNAs.

The EZ-QC™ XBG mRNA Capping Efficiency Assay Kit Contents are outlined in the following table:

EZ-QC ™ XBG mRNA Capping Efficiency Assay Kit Contents (10 reactions)
Sufficient for 10 experimental and 10 control reactions.

| | Reagent | |
|---|---|---|
| Kit Component | Volume | |
| EZ-QC ™ RNase H | 23 | μl |
| in 50% glycerol, 50 mM Tris-HCl, pH 7.5, 100 mM NaCl, 1 mM | | |
| dithiothreitol (DTT), 0.1 mM EDTA and 0.1% Triton ® X-100. | | |
| 10X EZ-QC ™ RNase H Reaction Buffer | 23 | μl |
| 0.2M Tris-acetate, pH 7.9, 0.5M potassium acetate, 0.1M magnesium | | |
| acetate and 0.01M DTT. | | |
| ScriptGuard ™ RNase Inhibitor, 40 U/μl | 12 | μl |
| in 50% glycerol, 50 mM Tris-HCl, pH 7.5, 100 mM NaCl, 10 mM DTT, | | |
| 0.1 mM EDTA and 0.1% Triton X-100. | | |
| XBG 5′ UTR Targeting Oligo, 5 μM (5 pmoles/μl) | 28 | μl |
| in RNase-Free Water. | | |
| XBG 5′ UTR Control Mix, 2.5 μM (2.5 pmoles/μl) | 6 | μl |
| an 80% capped/20% uncapped RNA Mix in RNase-Free Water. | | |
| Stop/Loading Buffer | 230 | μl |
| 95% formamide, 10 mM EDTA, pH 7.5, 0.01% Bromophenol Blue and | | |
| 0.01% Xylene Cyanol. | | |
| RNase-Free Water | 575 | μl |

As shown in FIG. 12, the resulting products were analyzed by polyacrylamide gel electrophoresis (PAGE), producing distinct bands corresponding to capped and uncapped species, enabling accurate quantification without cleanup or prior sequence knowledge.

As shown in FIG. 13, the same processed products were submitted for LC-MS analysis on an Orbitrap platform with deisotoping and charge deconvolution software, ProMass. The EZ-QC™ assay facilitated LC-MS compatibility without imposing a 13-23 nucleotide fragment size limitation or requiring removal of targeting oligonucleotides, proteins, or mRNA 3′ ends. PAGE-based detection provided rapid, direct visualization of capping efficiency, while LC-MS confirmed accuracy within +500 of the known input ratio, demonstrating the assay's robustness and workflow advantages over conventional LC-MS methods.

The LC-MS Results are outlined in the following table:

LC-MS Result

| | Observed | |
|---|---|---|
| RT (min) | Mass (Da) | Identity |
| 17.077 | 11367.385 | 10834.3792 |
| | | (m⁷g_cap1) |
| 17.077 | 11353.373 | 10834.3792 |
| | | (m⁷g_cap0) |
| 17.283 | 11074.271 | 10834.3792 |
| | | (uncapped triphosphate) |
| 17.077 | 10994.303 | 10834.3792 |
| | | (uncapped diphosphate) |
| 16.913 | 10914.33 | 10834.3792 |
| | | (uncapped phosphate) |

Table of relevant mRNA components found within the sample via LC-MS testing were identified based on mass to charge ratio (m/z) due to several molecules having similar retention times. Numerous species were detected that were irrelevant to the capping identity of the sample and only a subset is shown here.

As can be derived from the below table, which provides a comparison of the EZ-QC™ XBG mRNA Capping Efficiency Assay and LC-MS Results, comparable results were obtained between EZ-QC™ XBG mRNA Capping Efficiency Assay Kit and LC-MS.

Compiled EZ-QC ™ and LC-MS Results

| | EZ-QCTM | LC-MS |
|---|---|---|
| Sample | % Capped | % Capped |
| 80% Capped/20% Uncapped | 75.1% | Not performed |
| XBG RNA Control | | |
| 90% Capped/10% Uncapped | 89.1% | 93.0% |

The following key points are of relevance:
The method of determining the Capping efficiency of mRNA can be used for detection of cap-dependent and cap-independent products performed by polyacrylamide gel electrophoresis (PAGE), providing direct visualization of capping efficiency.
The enzymatically processed products may be optionally analyzed by LC-MS without additional cleanup to remove targeting oligonucleotides, proteins, or mRNA 3′ ends.
The calculated capping efficiency from PAGE analysis corresponds within +5% of the known input ratio confirmed by LC-MS analysis
The enzymatic processing enables LC-MS compatibility without imposing a 13-23 nucleotide fragment size limitation
The PAGE-based detection provides rapid discrimination of capped versus uncapped species compared to LC-MS workflows requiring prior knowledge of fragment lengths and sequences
The kit may comprise instructions which specify determining capping efficiency by PAGE analysis and optionally confirming accuracy by LC-MS analysis of the same processed products.
PAGE detection as the primary method of detection.
LC-MS compatibility is an optional confirmation step.
The methods of the invention provide Capture workflow advantages, including no size limit, no cleanup, no prior sequence knowledge.

### Procedures

A sample was submitted to Novatia, LLC., for the purpose of characterization of an mRNA using their LC-MS platform. Novatia utilizes liquid chromatography coupled to high resolution mass spectrometry and novel deisotoping and charge deconvolution software for characterization of mRNA caps and Poly A tails. Due to the large size and complexity of full-length mRNA, enzymatic processing of mRNA to cleave the 5′-capped end from full length mRNA was required for LC-MS testing. The EZ-QC™ XBG mRNA Capping Efficiency Assay was used to process the mRNA by cleaving the mRNA at the 5′ end for compatibility with LC-MS testing. The maximum fragment length of mRNA required for capping analysis was less than 35 nt using the LC-MS, and samples needed to be submitted already truncated. There was no maximum length restriction for mRNA processed using the EZ-QC™ XBG mRNA Capping Efficiency Assay, which could process long mRNA without restrictions. 5-10 picomoles were required for the EZ-QC™ XBG mRNA Capping Efficiency Assay, whereas 150-250 picomoles were required for LC-MS analysis.

The EZ-QC™ XBG mRNA Capping Efficiency Assay and the LC-MS method required the same initial sample-processing steps, but their downstream workflows diverged significantly. The EZ-QC™ XBG mRNA Capping Efficiency Assay generated material suitable for direct electrophoretic analysis without additional cleanup. In contrast, samples intended for LC-MS analysis required further purification using the Monarch Spin RNA Cleanup Kit to remove residual probe, buffer salts, and protein components—such as the RNase H enzyme—that could interfere with mass spectrometric performance. Although purification was not necessary for analysis by the EZ-QC™ XBG mRNA Capping Efficiency Assay alone, these samples were also purified to enable a consistent side-by-side comparison with the LC-MS workflow. No electrophoresis-associated dyes were added to samples prior to LC-MS analysis.

The LC-MS workflow employed the HRMS_LCMS method, which required the use of a specialized chromatographic column (Clarity Oligo-xt, 2.1×50 mm). In contrast, the EZ-QC™ XBG mRNA Capping Efficiency Assay did not require any specialized column hardware. The cost of analyzing a single sample by LC-MS was approximately 4-10 times higher than the cost of analyzing a sample using the EZ-QC™ XBG mRNA Capping Efficiency Assay. Throughput also differed: the EZ-QC™ kit enabled the simultaneous processing of approximately 10-15 samples per run, whereas LC-MS permitted evaluation of only one sample per analytical run. LC-MS required a system-suitability control for each run, while the EZ-QC™ kit did not. Instead, the EZ-QC™ XBG assay included an internal kit control consisting of an mRNA mixture with 80% capped and 20% uncapped transcripts to support accurate interpretation of capping efficiency.

The EZ-QC™ XBG mRNA Capping Efficiency Assay did not require prior knowledge of the expected molecular weight or length of the target mRNA. Capping efficiency was determined by resolving capped and uncapped mRNA species through standard electrophoretic separation followed by staining, as described in the kit protocol. In contrast, LC-MS analysis required advance knowledge of the expected molecular weights and relative abundances of the mRNA-derived moieties. This level of prior information was unnecessary for the EZ-QC™ XBG assay, which is suitable for both known and unknown samples.

Instrumentation requirements differed substantially between the two approaches. The EZ-QC™ XBG mRNA Capping Efficiency Assay required only benchtop electrophoresis equipment and a gel imager, typically costing approximately $5,000-$10,000. By comparison, an Orbitrap mass spectrometry platform required for LC-MS can cost several hundred thousand dollars to more than $1 million. Routine maintenance was necessary to ensure proper performance of the Orbitrap system, whereas electrophoresis systems and the Genesys imager required minimal upkeep.

Operational complexity also varied. The EZ-QC™ XBG assay relied on basic laboratory techniques and did not require specialized training to perform or interpret. LC-MS analysis, however, required expertise in instrument operation, use of analytical software, and interpretation of complex mass spectral data, particularly because many molecules can exhibit similar mass profiles, complicating analysis.

Workflow accessibility further distinguished the methods. The EZ-QC™ XB3G assay allowed for convenient and flexible in-house testing. In contrast, LC-MS analysis often required outsourcing, establishing accounts with external vendors, and shipping samples. It also required substantial instrument preparation and familiarity with specialized analysis software and data outputs. Turnaround time for the EZ-QC™ XBG assay was approximately one day, whereas LC-MS analysis required 1-5 days, not including shipping time.

Relevant information is tabulated below.

| Factor | EZ-QC ™ Assay Kits | LC-MS |
|---|---|---|
| Length of mRNA for | No max length restrictions | <35 nt is the max fragment length to |
| capping analysis | | achieve optimal analysis |
| Quantity of mRNA | 5-10 picomoles for Capping | 30-50 μl of a 5 μM solution = 150-250 |
| | efficiency kit | picomoles total required for each QC |
| | | analysis. |
| Turnaround time | Same day | >1-5 days, not including shipping time |
| Cost per sample | 4-10 samples for the cost of 1X | 1X LC-MS sample cost |
| | LC-MS sample | |
| Number of samples | 10-15 samples per gel, scalable | 1 sample per run |
| per run | based on needs | |
| Controls required | An 80% capped/20% uncapped | System suitability controls. Essential to |
| | control is included in the kit to | provide additional controls to run |
| | aid analysis | alongside unknowns. |
| Expected molecular | Not required | Yes, so the software can assign expected |
| weight or length of | | molecular weights and relative |
| target needed | | abundance. |
| for analysis | | |
| Equipment required | Benchtop electrophoresis and | LC-MS platform |
| | gel imager | |
| Training required | Basic lab techniques | Yes, specialized training is required for |
| | | using the software, interpreting data and |
| | | using the equipment. Many molecules |
| | | have similar profiles, making analysis |
| | | difficult. |
| Flexibility of | Process and analyze samples | Requires possible outsourcing of |
| testing | when convenient | testing, account setup, and scheduling |
| | | of shipment on dry ice. |
| | | Requires up-front prep of platform and |
| | | knowledge of the analysis software and |
| | | data output. |

### REFERENCES

1. Analytical Procedures for Quality of mRNA Vaccines and Therapeutics (Draft Guidelines: 3rd Edition)”, 2 Aug. 2024, US Pharmacopeia Expert Committee: Biologics Monograph 3—Complex Biologics & Vaccines https://www.uspnf.com/notices/analytical-procedures-mrna-vaccines-20240802
2. McKenzie R E et al. “mRNA Synthesis and Encapsulation in Ionizable Lipid Nanoparticles”, Curr Protoc. 2023 September; 3(9):e898. doi: 10.1002/cpz1.898
3. Inoue et al., “Sequence-dependent hydrolysis of RNA using modified oligonucleotide splints and RNase H”, FEBS Lett. 1987 May 11; 215(2):327-30. doi: 10.1016/0014-5793(87)80171-0
4. Lapham, J and Crothers, D M, 1996, “RNase H cleavage for processing of in vitro transcribed RNA for NMR studies and RNA ligation”, Rna, 2(3), 289-296
5. Lapham et al., “The position of site-directed cleavage of RNA using RNase H and 2′-O-methyl oligonucleotides is dependent on the enzyme source”, RNA, 1997 September; 3(9):950-1”
6. U.S. Pat. No. 11,365,437B2
7. Tu et al., 2024, “Capped or uncapped? Techniques to assess the quality of mRNA molecules”, Current Opinion in Systems Biology, 100503. Other embodiments include the use of an alternative RNase, e.g. RNase 4 in place of RNase H
8. Masahide Ishikawa, et al, Preparation of eukaryotic mRNA having differently methylated adenosine at the 5′-terminus and the effect of the methyl group in translation, Nucleic Acids Symposium Series, Vol 53, Issue 1, September-October 2009, Pages 129-130, doi.org/10.1093/nass/nrp065 and references therein, which are incorporated herein by reference in their entirety.
9. WIPO PCT WO2007120863A2, which is incorporated herein by reference in its entirety.

1.-17. (canceled)

18. A method of quantitating the relative amounts or percentages of RNA molecules that are 5′-capped and that are uncapped in an RNA sample or for quantifying the efficiency of capping of an in vitro-synthesized mRNA of interest that has been subjected to a capping enzyme system, said method comprising the following steps a) to e):
a) providing an RNA sample, wherein said sample comprises:
i) mRNA with a cap structure at the most 5′-end ribonucleotide of said mRNA of interest; and
ii) mRNA without said cap structure at the most 5′-end ribonucleotide of said mRNA of interest;
b) contacting said RNA sample with an 5′RNA-DNA-RNA3′ targeting oligonucleotide consisting of a DNA central portion flanked at its 5′-end and its 3′-end by RNA portions, under conditions that permit said targeting oligonucleotide, including said DNA central portion, to hybridize to said mRNA of interest to form a hybrid between the mRNA of interest and the targeting oligonucleotide, wherein said targeting oligonucleotide is further defined as follows:
i) said DNA central portion comprises or consists of four deoxyribonucleotides in length, wherein said DNA central portion is complementary to a sequence at the 5′-end portion of said mRNA of interest within 15 to 50 bases from said most 5′ end ribonucleotide; and
ii) each of said RNA flanking portions comprises 5 to 15 ribonucleotides in length and said RNA flanking portions are complementary to corresponding RNA portions in said mRNA of interest flanking the sequence hybridized to the targeting oligonucleotide's DNA central portion;
c) providing a nuclease that selectively cleaves RNA in a DNA/RNA hybrid, resulting in the cleavage of said mRNA of interest in the sequence region hybridized to the targeting oligonucleotide's DNA central portion and the release of:
i) the cleaved mRNA 5′-end portion fragments with said cap structure; and
ii) the cleaved mRNA 5′-end portion fragments without said cap structure;
d) electrophoretically separating said released mRNA 5′-end portion fragments; and
e) determining the relative amount of said separated capped fragments and uncapped fragments, thereby quantifying efficiency of mRNA capping in said mRNA of interest.

19. A method of quantitating the relative amounts or percentages of RNA molecules that are 5′-capped and that are uncapped in a sample or for quantifying the efficiency of mRNA capping of an in vitro-synthesized RNA of interest that has been subjected to a capping enzyme system, said method comprising the following steps a) to e):
a) providing an RNA sample, wherein said sample comprises:
i) mRNA with a cap structure at the most 5′-end ribonucleotide of said mRNA of interest; and
ii) mRNA without said cap structure at the most 5′-end ribonucleotide of said mRNA of interest;
b) contacting said mRNA sample with an 3′RNA-DNA5′ targeting oligonucleotide consisting of a 3′ RNA portion and a 5′ DNA portion, under conditions that permit said targeting oligonucleotide, including said DNA portion, to hybridize to said mRNA of interest to form hybrid between an mRNA of interest and a targeting oligonucleotide, wherein said targeting oligonucleotide is further defined as follows:
i) said DNA portion comprises or consists of four deoxyribonucleotides in length, wherein said DNA portion is complementary to a sequence at the 5′-end portion of said mRNA of interest within 15 to 50 bases from said most 5′ end ribonucleotide,
ii) said RNA portion comprises 5 to 15 ribonucleotides in length and said RNA portion is complementary to a corresponding RNA portion in said mRNA of interest flanking the sequence hybridized to the targeting oligonucleotide's DNA portion;
c) providing a nuclease that selectively cleaves RNA in a DNA/RNA hybrid, resulting in the cleavage of said mRNA of interest in the sequence region hybridized to the targeting oligonucleotide's DNA portion and the release of:
i) the cleaved mRNA 5′-end portion fragments with said cap structure; and
ii) the cleaved mRNA 5′-end portion fragments without said cap structure;
d) electrophoretically separating said released mRNA 5′-end portion fragments; and
e) determining the relative amount of said separated capped fragments and uncapped fragments, thereby quantifying efficiency of mRNA capping in said mRNA of interest.

20. A method of quantitating the relative amounts or percentages of RNA molecules that are 5′-capped and that are uncapped in a sample or for quantifying the efficiency of RNA capping of an in vitro-synthesized RNA of interest that has been subjected to a capping enzyme system, said method comprising the following steps a) to e):
a) providing an RNA sample, wherein said sample comprises:
i) mRNA with a cap structure at the most 5′-end ribonucleotide of said mRNA of interest; and
ii) mRNA without said cap structure at the most 5′-end ribonucleotide of said mRNA of interest;
b) contacting said mRNA sample with a 5′RNA-DNA3″ targeting oligonucleotide consisting of a 5′ RNA portion and 3′ DNA portion, under conditions that permit said targeting oligonucleotide, including said DNA portion having at least on ribonucleotide, to hybridize to said mRNA of interest to form a hybrid between the mRNA of interest and the targeting oligonucleotide, wherein said targeting oligonucleotide is further defined as follows:
i) said DNA portion having at least on ribonucleotide comprises or consists of four to six deoxyribonucleotides in length, wherein said DNA portion having at least on ribonucleotide is complementary to a sequence at the 5′-end portion of said mRNA of interest within 15 to 50 bases from said most 5′ end ribonucleotide; and
ii) said RNA portion comprises 5 to 15 ribonucleotides in length and said RNA portion is complementary to a corresponding RNA portion in said mRNA of interest flanking the sequence hybridized to the targeting oligonucleotide's DNA portion having at least on ribonucleotide, wherein said at least one ribonucleotide is complementary to the counterpart ribonucleotide of the mRNA of interest;
c) providing a nuclease that selectively cleaves RNA in a DNA/RNA hybrid, resulting in the cleavage of said mRNA of interest in the sequence region hybridized to the targeting oligonucleotide's DNA portion having at least on ribonucleotide and the release of:
i) the cleaved mRNA 5′-end portion fragments with said cap structure; and
ii) the cleaved mRNA 5′-end portion fragments without said cap structure;
d) electrophoretically separating said released mRNA 5′-end portion; and
e) determining the relative amount of said separated capped fragments and uncapped fragments, thereby quantifying efficiency of mRNA capping in said mRNA of interest.

21. The method of claim 1, wherein said mRNA of interest is produced by in vitro transcription (IVT) of a DNA template encoding said mRNA of interest, and wherein said cap structure is post- or co-transcriptionally added to the most 5′-end ribonucleotide of said mRNA of interest.

22. The method of claim 21, wherein said IVT produced mRNA (1a) with said cap structure comprises an additional N7-methylated guanosine linked by a 5′ to 5′ triphosphate bridge to the 5′-end of said IVT mRNA as shown below 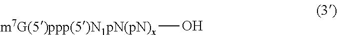
wherein:
m⁷G represents the N7-methylguanosine cap nucleoside,
ppp represents the triphosphate bridge between the 5′ carbons of the cap nucleoside and the first ribonucleotide of the primary RNA transcript, and
N₁pN(pN)ₓ—OH (3′) represents the primary RNA transcript, of which N₁ is the most 5′-end ribonucleotide.

23. The method of claim 1, wherein said nuclease only cleaves RNA that is hybridized to DNA and said nuclease has a minimum binding domain size of four nucleotides.

24. The method of claim 23, wherein said nuclease is RNase H.

25. The method of claim 1, wherein said nuclease cleaves said mRNA of interest only once.

26. The method of claim 1, wherein said electrophoretically separating comprises use of a gel that is a polyacrylamide gel selected from the group consisting of a polyacrylamide gel comprising 17% to 22% acrylamide, 20% acrylamide, and 19.5% acrylamide and 0.50% Bis-acrylamide.

27. The method of claim 1, wherein said released mRNA 5′-end portion fragments are fragments of 25 to 40 nucleotides in length.

28. The method of claim 1, wherein said targeting oligonucleotide is further defined by one of the following features or any combination(s) thereof:
i) the Tₘ of said targeting oligonucleotide (7) is 40° C. to 68° C.;
ii) the DNA portion of the targeting oligonucleotide is flanked with RNA portions on both ends;
iii) the targeting oligo (TO) anneals to an mRNA having an autologous 5′-untranslated region (5′ UTR);
iv) the targeting oligo (TO) anneals to a heterologous 5′-untranslated region (5′ UTR) that is used for many different mRNAs;
v) the targeting oligo anneals to a Xenopus 5′ UTR;
vi) said targeting oligo is designed to so the DNA portion does not hybridize to counterpart ribonucleotides in sequence at the 5′-end portion of the mRNA of interest that comprise modified ribonucleosides selected from pseudouridine and N1-methylpseudouridine; and
vii) the length of the targeting oligonucleotide in terms of nucleotides is different from the released mRNA 5′ end portion fragments.

29. The method of claim 1, said method further comprising:
a) providing:
i) a collection of molecular weight markers, including a marker ladder; and/or
ii) a control mixture of 5′-end capped and uncapped control mRNA fragments;
b) parallel electrophoresing: said released mRNA 5′-end portion fragments; and said collection of molecular weight markers and/or said control mixture of 5′-end capped and uncapped control mRNA fragments; and
c) comparing the length of the gel separated mRNA 5′-end portion fragments with said weight markers and/or said control mixture of 5′-end capped and uncapped control mRNA fragments.

30. A kit or kit-of-parts, said kit or kit-of-parts comprising:
a) a nuclease that selectively cleaves RNA in a DNA/RNA hybrid, and
b) a Stop/Loading buffer containing at least one dye or two dyes.

31. The kit or kit-of-parts of claim 30, wherein said nuclease is RNase H.

32. The kit or kit-of-parts of claim 30, further comprising bromophenol blue and xylene cyanol electrophoresis markers.

33. The kit or kit-of-parts of claim 30, further comprising an electrophoresis gel, wherein said electrophoresis gel selected from the group consisting of a polyacrylamide gel comprising 17% to 22% acrylamide, 20% acrylamide, 19.5% acrylamide and 0.50% Bis-acrylamide, and 20% acrylamide/8 M Urea/1×TBE.

34. The kit or kit-of-parts of claim 30, further comprising:
a. a collection of molecular weight markers; and/or
b. a control mixture of 5′-end capped and uncapped control mRNA fragments.

35. The kit or kit-of-parts of claim 34, wherein said control mixture comprises mRNA fragments comprising a ratio in the range of 9:1 to 1:9, including 3:2, 3:1, 1:1, 2:3, 1:3, of capped to uncapped fragments of the same mRNA fragment.

36. The kit or kit-of-parts of claim 30, further comprising an RNA staining dye.

37. The kit of kit-of-parts of claim 36, wherein said RNA staining dye comprises ethidium bromide, GelRed™, SYBR-GOLD and/or oxazole gold.
